(19) United States
(12) Patent Application Publication
(10) Pub. No.: US 2006/0141443 A1
(43) Pub. Date: Jun. 29, 2006

(54) Apparatus and method for transcellular testing

(76) Inventors: J. Richard Gyory, Sudbury, MA (US); Michael Cima, Winchester, MA (US); Javier Gonzalez-Zugasti, North Billerica, MA (US); Anthony Lemmo, Sudbury, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/542,477

(22) PCT Filed: Jan. 20, 2004
(86) PCT No.: PCT/US04/01275

§ 371 Date: Jul. 15, 2005

(30) Foreign Application Priority Data
Jan. 21, 2003 (US) 60441358

(51) Int. Cl. C12Q 1/00; C12M 1/34

(52) U.S. Cl. 435/4; 435/287.1

(57) ABSTRACT

The present invention relates to a device and method for facilitating high throughput transcellular flux testing of compounds, such as pharmaceuticals or drugs, other compounds, or compound combinations. In one embodiment, the system and methods of the present invention may be used to identify the optimal components (e.g., solvents, carriers, transport enhancers, adhesives, additives, inhibitors, or other excipients) for pharmaceutical compositions or formulations that are delivered to a patient via tissue transport, including without limitation, pharmaceutical compositions or formulations administered or delivered transcellularly, topically, and ocularly.

### RELATED APPLICATIONS

This application claims the benefit of U.S. Provisional Application No. 60/441,358 filed Jan. 21, 2003.

### TECHNICAL FIELD OF THE INVENTION

Generally this invention relates to a device and method for in vitro testing. More specifically, this invention relates to a device and method for high throughput transcellular testing.

### DESCRIPTION OF RELATED ART

Currently, there are numerous incurable diseases and new diseases and new forms of diseases are being discovered often. Accordingly, research and development of new and more effective drugs and pharmaceuticals is highly important. One important aspect of drug and pharmaceutical research and development relates to methods for delivering or administering drugs into a patient.

Traditional routes of drug administration include inhalation, intranasal, oral, rectal, vaginal, topical, infusion, and injection. A relatively recent advancement in drug administration is the administration of a drug directly across the skin of a patient, otherwise known as transdermal drug delivery. Typically, in transdermal delivery a drug is positioned on the outermost layer of a patient's skin (epidermis) and thereafter transfers through the skin and into the patient's body, typically via simple diffusion.

Transdermal delivery is a desirable delivery technique as it offers many advantages over other methods of drug delivery. For example, an advantage over injection delivery is the reduction of contamination and the ease of disposal, as compared to traditional syringe needles. In addition, the unpleasantness of receiving injections is avoided, leading to improved patient compliance of drug regimens. Furthermore, transdermal delivery is particularly useful for drugs that require repeated administration, such as insulin for diabetes, or the like.

Another advantage of transdermal drug delivery is the ability to maintain a constant drug concentration within the body over a long period of time, such as several days or weeks. Other delivery methods, such as oral or pulmonary delivery, typically require the drug to be administered repeatedly to sustain the proper drug concentration within the body. With traditional drug delivery methods concentration of the drug in the body spikes to a high level shortly after administration. These “spikes” can cause toxicity problems, thereby, making some otherwise viable drugs a less preferred treatment option. Unlike traditional drug delivery, transcellular drug delivery delivers a substantially constant flow of drug to the body over an extended period of time from days to weeks, thereby reducing the toxicity problems.

Another advantage of drugs administered using transdermal delivery is that they bypass the first-pass metabolism in the liver and avoid other degradation pathways such as the low pH's and enzymes present in the gastrointestinal tract. These biological barriers are avoided because transdermally administered drugs diffuse through the skin and directly into the blood stream without passing through the gastrointestinal tract.

An example of a transdermal drug delivery system currently in use is the D-Trans® system made by ALZA Corp. Mountain View, Calif. The D-Trans® system typically incorporates a series of thin, flexible films which include a backing layer, a drug reservoir, a rate-controlling film, and an adhesive. One drug this system is effective in delivering is nicotine. Two ALZA Corp. products, NicoDerm® CQ® and Clear NicoDerm® CQ® deliver nicotine to patients through the D-Trans® system to control nicotine withdrawal incurred by individuals attempting to quit smoking. In use, the nicotine stored in the drug reservoir transfers through the rate-controlling film and is absorbed or permeates through the skin of the user and into the blood stream.

However, transdermal drug delivery has its drawbacks. For example, it is difficult to transfer the drug across the epidermis of the skin of a patient. The skin is the largest organ of the body and is naturally highly impermeable to prevent loss of water and electrolytes and to prevent the body from being invaded by foreign substances such as bacteria, viruses, liquids, and other compounds and materials. Accordingly, the natural barrier to permeability, the skin, also severely restricts the potential for transdermal delivery to a wide array of drugs.

The skin is generally subdivided into two main layers: the outer layer being the epidermis and the inner layer being the dermis. The epidermis is about 50 to 100 micrometers thick. The dermis varies from 1 to 3 millimeters in thickness. The blood capillaries are housed in the dermis and, therefore, it is the goal of transdermal drug delivery to get the drug to cross the epidermis and enter the dermis such that the drug can enter the blood stream for systemic delivery.

The epidermis is categorized into several layers. The outermost layer of epidermis is called the stratum corneum. The stratum corneum is comprised of dead cells called corneocytes or keratinocytes. The stratum corneum is commonly modeled or described as a brick wall. The “bricks” are the flattened, dead corneocytes. Typically, there are about 10 to 15 corneocytes stacked vertically across the stratum corneum. The corneocytes are encased in sheets of lipid bilayers (the “mortar”). The lipid bilayer sheets are separated by approximately 50 nm. Typically, there are about 4 to 8 lipid bilayers between each pair of corneocytes. The lipid matrix is primarily composed of ceramides, sphingolipids, cholesterol, fatty acids, and sterols, with very little water present.

Although it is the thinnest layer of the skin, the stratum corneum is the primary barrier to entry of molecules or microorganisms into the body. Once the molecules have crossed the stratum corneum, diffusion across the remaining layers of the epidermis and dermis to the blood vessels occurs rapidly. Thus, most of the attention in transdermal drug delivery research has been focused on transporting molecules and drugs across the stratum corneum.

Another drawback of transdermal drug delivery is that currently, the technique is effective only with small, lipophilic molecules which readily permeate the skin. However, many substances have been developed to enhance the molecular transport rates of less permeable drugs. These substances are known as chemical enhancers or penetration enhancers. Chemical enhancers attempt to increase the flux of a drug through the skin by increasing the solubility of a drug in the stratum corneum or by increasing the permeability of the drug in the stratum corneum.

Selecting a proper enhancer is both difficult and complicated, as there is a myriad of possible enhancer/drug combinations. Not all enhancers are suitable for use with all drug molecules as some might interact with the drug molecule and cause an undesirable effect within the body. Further, some combinations of enhancers may improve drug flux beyond the expected flux rate and therefore result in too high of a drug concentration being delivered over too short of an interval for effective or safe treatment.

A further drawback is the adhesive used with transdermal delivery patches. The adhesive is required to keep the patch in place on a patient, however, there are many different forms of adhesives that can be used. Typically, it is difficult to select which adhesive to use with any particular drug, and/or drug and enhancer combination, because there may be a chemical interaction between the various chemical compounds.

Currently, the choice of appropriate enhancers, adhesives, and their relative proportion with respect to the drug is determined by general guidelines from what is known to be safe and what may have been effective with other drugs. The vast majority of the formulation development is made through trial and error experimentation, as the current transdermal testing devices are inadequate.

To date, the devices for transdermal testing are relatively large, inefficient, ineffective, costly, and prone to error. In particular, two types of transcellular testing devices are currently in use, the Ussing chamber and the “filter insert” device. These devices will be briefly explained along with their associated drawbacks.

The Ussing chamber, invented by Dr. Hans Ussing is configured for transcellular testing and consists of two hemi-chambers or reservoirs with open ends that are separated by a tissue or cellular layer located on a permeable membrane. In use, the two open ended reservoirs are clamped together with the tissue layer on the permeable membrane pinched between the reservoirs. One reservoir, the testing reservoir, is filled with a particular solution containing some drug or pharmaceutical composition (with or without enhancers or other additives) and the other reservoir, the sampling reservoir, is filled with a neutral solution, such as a saline type solution. Over a given time interval, samples are withdrawn from the sampling reservoir to determine what compounds, if any have diffused from the testing reservoir, across the tissue layer, and into the sampling reservoir.

The Ussing chamber system, however, has several drawbacks. The Ussing chamber is not compatible with high throughput testing regimes and, therefore, amenable to testing only a handful of compounds or substances. This is unacceptable given the myriad of drug, enhancer, adhesive, or the like components and combinations of these components that require testing. As a result, testing only a few samples at any particular time is both inefficient and ineffective. Furthermore, the relatively large dimensions of the Ussing chamber device require a large amount of laboratory space, many technicians, and a large quantity of resources. This substantially increases the cost and time required to conduct the necessary testing of new drug delivery compositions.

Another drawback of the Ussing chamber device comes about while clamping the membrane, with the cellular layer thereon, between the reservoirs. When the two reservoirs are clamped together damage frequently occurs to the tissue or cellular layer. The damage most often occurs near the edges of the reservoir, where the reservoirs pinch the cellular layer together to form a tight seal. This clamping damage typically produces a “gap” in the cellular layer between the abutting reservoirs. This “gap” functions as an open passage through which the compounds in each reservoir may freely transfer, thereby bypassing transfer through the cellular layer and compromising the experiment results. Therefore, a high throughput transcellular testing device would be highly desirable.

The “filter insert” device currently in use consists of a sleeve or cylindrical tube where one end of the sleeve is closed off by a permeable membrane with a cellular layer grown across it. The other end of the sleeve is left open for receiving, sampling, or depositing substances. In use, the sleeve is placed, cellular end first, into a reservoir containing a sample solution. The sample in the reservoir then diffuses through the cellular layer comprising the end of the sleeve, and into the sleeve. Sampling and subsequent analysis of the resulting composition in the sleeve is used to determine the diffusion and flux rate through the cellular layer.

However, the “filter insert” device has many of the same disadvantages of the Ussing chamber. First, the “filter insert” device is not compatible with high throughput and requires large quantities of testing materials. Second, the cellular layer used in the device is grown over a permeable membrane and a junction where the permeable membrane attaches to the sleeve. At the permeable membrane/sleeve junction there is often incomplete cellular growth creating “gaps” between the reservoir and the sleeve where the substance can freely pass into the sleeve without diffusing through the cellular layer.

Furthermore, as a natural characteristic of cell cultures, the cellular layer does not grow uniformly across different base materials. Therefore, because the cellular layer is not uniform, the rate of transport or diffusion is not a constant throughout the cellular layer and any flux rate calculation not accounting for this will be flawed.

Like the Ussing device, the “filter insert” configuration requires costly equipment and space, multiple operators to perform the desired experiments, and is also prone to error. As a result, innovation related to transdermal drug delivery compositions has been delayed. In light of the above, a device and method that addresses the above described drawbacks would be highly desirable. Specifically a device that can facilitate accurate high-throughput testing of transcellular drug flux would be highly desirable.

### SUMMARY OF THE INVENTION

According to the invention there is provided a device for accurate high throughput testing of transcellular drug flux.

The present invention relates to a device and method for facilitating high throughput transcellular flux testing of compounds, such as pharmaceuticals or drugs, other compounds, or compound combinations. In one embodiment, the system and methods of the present invention may be used to identify the optimal components (e.g., solvents, carriers, transport enhancers, adhesives, additives, inhibitors, or other excipients) for pharmaceutical compositions or formulations that are delivered to a patient via tissue transport, including without limitation, pharmaceutical compositions or formulations administered or delivered transcellularly (e.g., in the form of a transcellular delivery device), topically (e.g., in the form of ointments, lotions, gels, and solutions), and ocularly (e.g., in the form of a solution). As used herein, “high throughput” refers to the number of samples generated or screened as described herein, typically at least 10, more typically at least 50 to 100, and preferably more than 1000 samples tested simultaneously in the same device.

The transcellular testing device of the present invention addresses the drawbacks of current devices and methods, because it does not damage the cellular layer, contains a uniform cellular layer that produces uniform diffusion rates, and can be used for high throughput testing. The transcellular testing device of the present invention does not damage the cellular layer because there is no clamping or crushing of the cellular layer between components of the device. Therefore there are no gaps in the cellular layer, through which the testing substances can freely flow without being diffused through the cellular layer. This results in more accurate results than the traditional methods. Furthermore, there is no variation in the cellular layer because the surface on which the cellular layer is grown is uniform. This produces uniform transportation or diffusion across the entire cellular layer and yields more accurate results than traditional methods. Finally, the transcellular testing device is capable of use in high throughput testing. This results in the ability to test multiple variations of compounds and substances in a rapid manner, thereby resulting in an increased number of suitable transcellularly administered drugs.

In one aspect, the present invention incorporates a transcellular testing device, comprising:
(a) a membrane having a first membrane surface opposing a second membrane surface, wherein said first membrane surface is configured for cellular adhesion; and
(b) a hydrophobic layer having a first hydrophobic layer surface opposing a second hydrophobic layer surface wherein said second hydrophobic layer surface is coupled to said second membrane surface, and wherein said hydrophobic layer comprises at least one discrete independent testing unit having;
   (i) a first opening in said first hydrophobic layer surface;
   (ii) a second opening in said second hydrophobic layer surface; and
   (iii) a passageway defined by said hydrophobic layer between said first opening and said second opening.

### BRIEF DESCRIPTION OF THE DRAWINGS

The features and advantages of the present invention will be better understood by reference to the following detailed description, which should be read in conjunction with the accompanying drawings in which:

FIG. 1 is an oblique view of a modified membrane according to the present invention;

FIG. 2A is a cross sectional view of a transcellular testing device, according to an embodiment of the invention, as taken along the line X-X′ of FIG. 1;

FIG. 2B is a cross sectional view of another transcellular testing device, according to another embodiment of the invention, as taken along the line X-X′ of FIG. 1;

FIG. 2C is a cross sectional view of yet another transcellular testing device, according to yet another embodiment of the invention, as taken along the line X-X′ of FIG. 1;

FIG. 2D is a cross sectional view of even another transcellular testing device, according to even another embodiment of the invention, as taken along the line X-X′ of FIG. 1;

FIG. 2E is a cross sectional view of a further transcellular testing device, according to a further embodiment of the invention, as taken along the line X-X′ of FIG. 1;

FIG. 2F is a cross sectional view of still another transcellular testing device, according to still another embodiment of the invention, as taken along the line X-X′ of FIG. 1; and

FIG. 3 is a flow chart of a method for making and using a transcellular testing device, according to the present invention.

Like reference numerals refer to corresponding parts throughout the views of the drawings.

### DETAILED DESCRIPTION OF THE PREFERRED EMBODIMENT

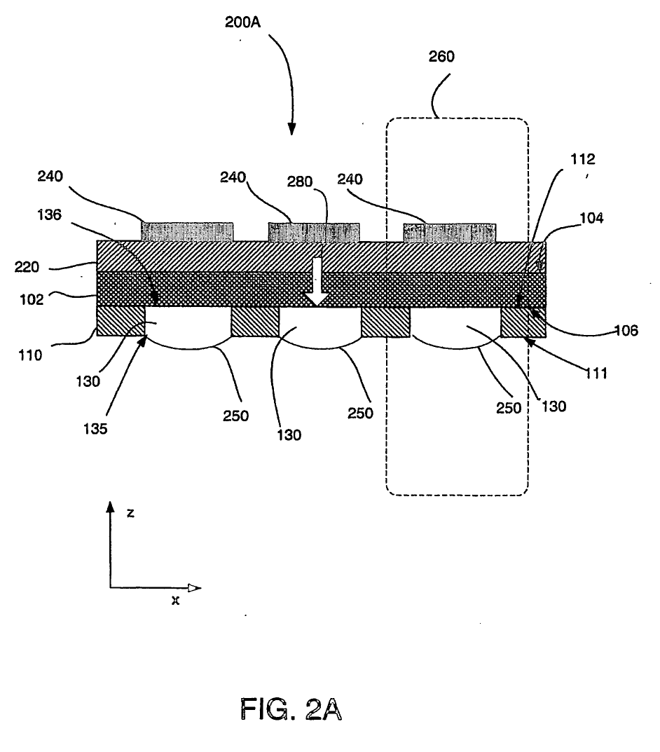

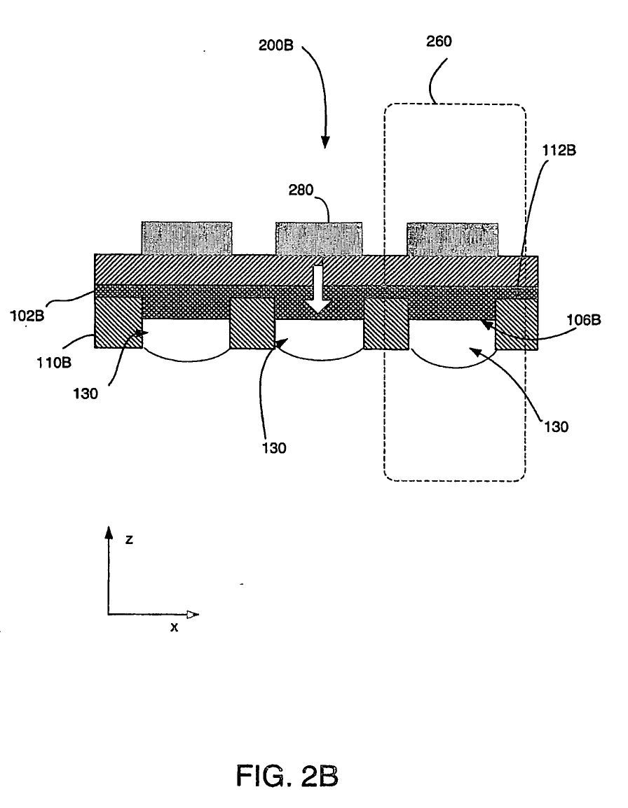

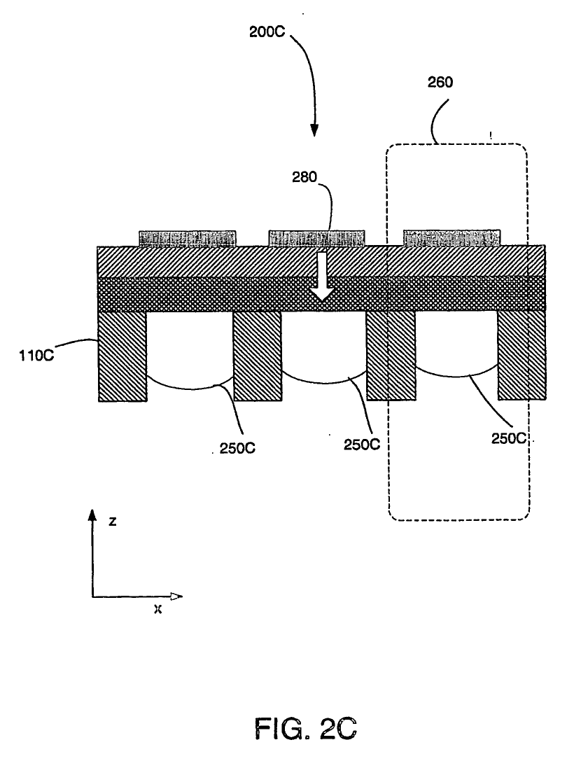

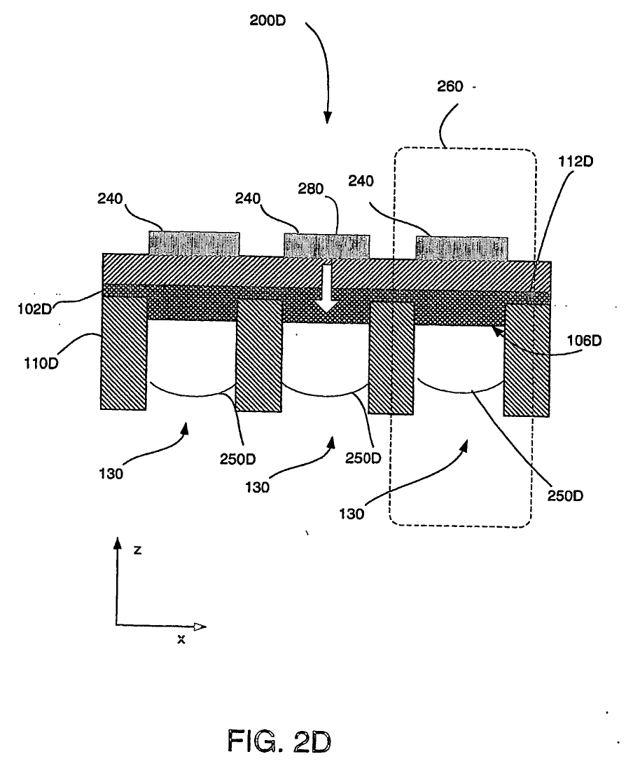

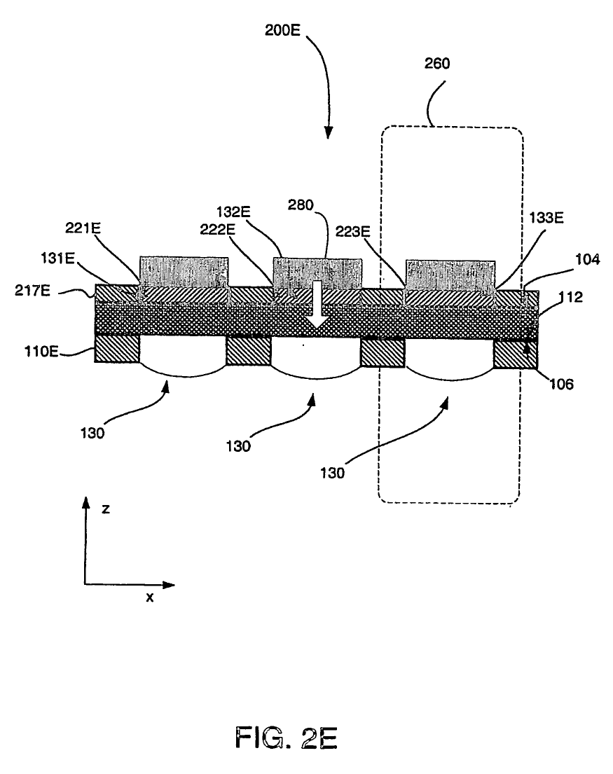

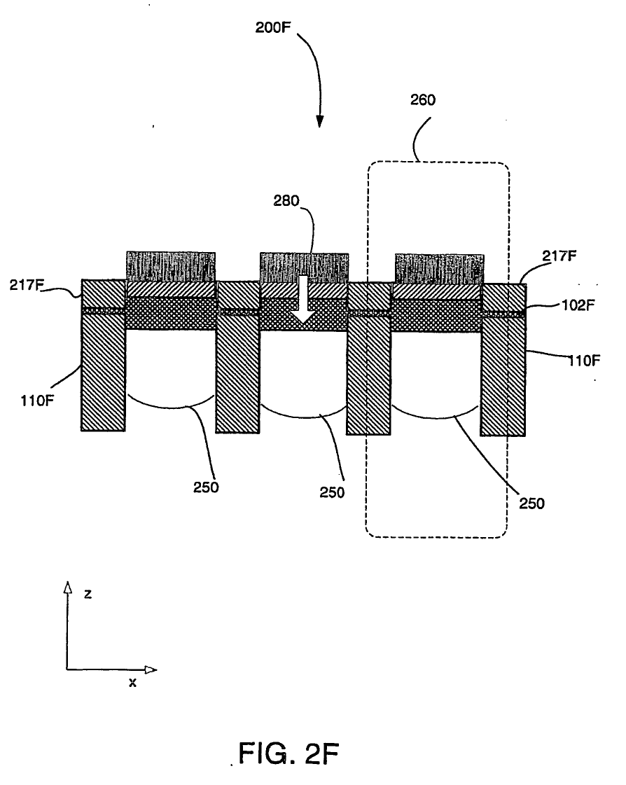

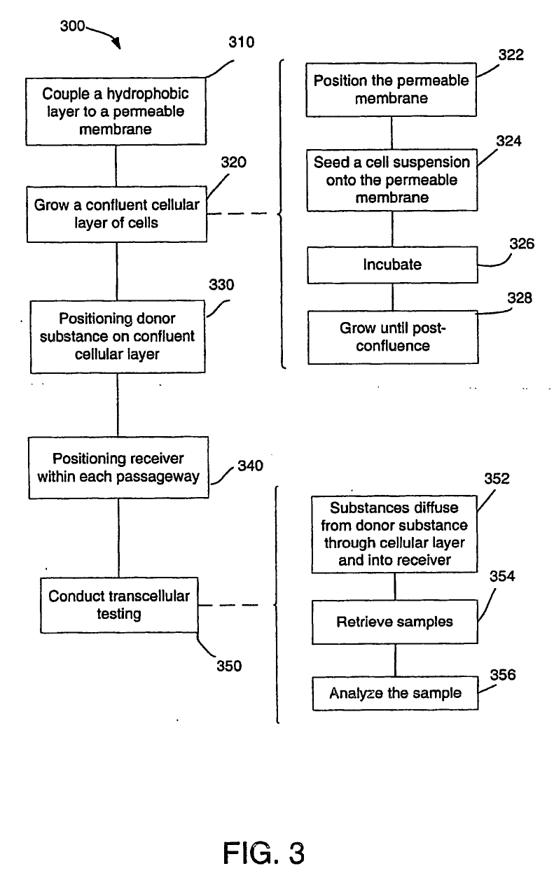

Transcellular testing devices 200A-200F (FIG. 2A-2F) of the present invention preferably comprise a permeable membrane (hereinafter “membrane”), a cellular layer, a hydrophobic layer with passageways through it, donor substances, and receivers. In use, chemical substances including drug molecules, pharmaceuticals, enhancers, adhesives, and other additives positioned in the donor substance, diffuse out of the donor substance, through the cellular layer, through the membrane, and into the receivers. Samples are taken from the receivers and analyzed to determine the composition of the chemical substances that have diffused across the cellular layer. Also, the flux rate for the tested composition is determined. Further details describing the making and using of the transcellular testing devices 200A-200F can be found below in relation to FIG. 3.

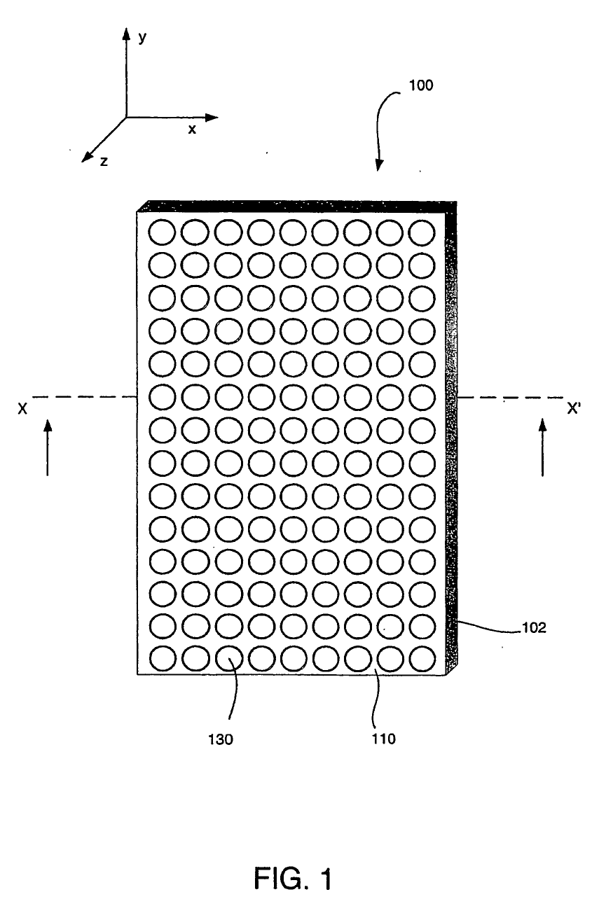

FIG. 1 shows a modified membrane 100 of the transcellular testing devices 200A-200F (FIG. 2A-2F) according to an embodiment of the present invention. In this configuration, a membrane 102 is bound to a hydrophobic layer 110. The hydrophobic layer 110 forms an array of passageways 130 for transcellular testing. Further details of the structure of the transcellular testing devices 200A-200F can be found below. It will be appreciated by those skilled in the art that the array of passageways 130 can be manufactured to inter-operate with standard testing or dispensing machinery, such as machinery used in conjunction with standard microtiter plates.

FIG. 2A-2F are cross sections of different embodiments of the transcellular testing devices 200A-200F, as taken along line X-X′ of FIG. 1. The transcellular testing devices 200A-200F generally include the membrane 102 and the hydrophobic layer 110 defining passageways 130 therethrough, as described below. Initially, the transcellular testing devices 200A-200F will be described in detail with respect to FIG. 2A, thereafter, alternative embodiments will be described in detail.

FIG. 2A shows a transcellular testing device 200A according to an embodiment of the invention. The transcellular testing device 200A includes a membrane 102 having a first membrane surface 104 opposing a second membrane surface 106. It is preferable that the first and second membrane surfaces are substantially planar. The membrane 102 is any hydrophilic porous membrane typically used in medical or pharmaceutical laboratories. Suitable examples of the membrane 102 are the HAWP membrane filter made by Millipore Corp. of Massachusetts; the Nuclepore® polycarbonate membrane filters made by Whatman of Massachusetts; or the Cyclopore® membrane made by Becton Dickinson of New Jersey.

The membrane 102 is preferably between about 40 and about 200 mm wide (as taken along the x axis) and about 50 and about 300 mm long (as taken along the y axis, shown in FIG. 1). However, in a preferred embodiment the membrane 102 has substantially the same width and length as a standard microtiter plate, and is preferably between about 25 micrometers and about 2 mm thick (along the z axis), and more preferably about 0.15 mm thick. For example, a width of about 85.5 mm and a length of about 127.8 mm are preferred membrane dimensions.

The membrane 102 is also porous, thereby allowing fluids and particles smaller than the membrane's pore size to pass therethrough. In a preferred embodiment, the pore size of the membrane 102 is dependent upon the size of pores required for cellular growth and adherence to the membrane 102. An example of pore size that effectuates cell growth and adherence is pores between about 0.1 micrometers and about 10 micrometers in diameter, and more preferably about 0.1 to about 5 micrometers.

In another embodiment, the membrane 102 can be any material that is permeable to fluids, whether or not it is able to sustain cellular growth and attachment. However, in this embodiment, the first membrane surface 104 is treated with a substance that promotes cellular growth and adhesion. For example, the first membrane surface 104 may be treated with a substance that has a rough or porous structure and has properties that are compatible with cellular growth and adhesion. For example, the first membrane surface 104 may be treated with a variety of biologically compatible materials, such as collagen type I, or the like.

The second membrane surface 106 is treated with a hydrophobic layer 110. The hydrophobic layer 110 is any hydrophobic, biologically and chemically inert material. A suitable hydrophobic layer 110 is TEFLON® made by Dupont. Other suitable materials include wax, polypropylene, polyethylene, polyvinyl chloride (PVC), or the like.

The hydrophobic layer 110 has a first hydrophobic layer surface 111 and an opposing second hydrophobic layer surface 112. The hydrophobic layer 110 preferably has at least the same width and length as the membrane 102. The hydrophobic layer 110 is also preferably substantially planar.

The second hydrophobic layer surface 112, is coupled to the second membrane surface 106 by any of several processes including, but not limited to, heat bonding, adhesive attachment, vapor deposition, or the like. A preferred process of coupling the hydrophobic layer 110 to the membrane 102 is by heat sealing parafilm to the second membrane surface 106.

The first hydrophobic layer surface 111 has at least one first opening 135 (FIG. 2A). The second hydrophobic layer surface 112 has at least one second opening 136 (FIG. 2A). The first opening 135 and the second opening 136 are connected to each other, forming a passageway 130 through the hydrophobic layer 110, such that the passageway runs parallel to the z-axis. Accordingly, once the hydrophobic layer 110 is coupled to the second membrane surface 106, permeability through the transcellular testing device 200A is restricted to the passageways 130 through the hydrophobic layer 110.

In a preferred embodiment, the hydrophobic layer 110 defines an array of passageways 130. The number of passageways 130 varies depending of the surface area of the membrane 102, the diameter of the passageways 130, and the distance between the passageways 130. In a preferred embodiment the array of passageways 130 are configured to mate with a standard array pin replicator or multi-channel pipettor such as those used to dispense fluids into a microtiter plate. In a preferred embodiment of the present invention, there are 24, 96, 384, or 1536 passageways 130 through the hydrophobic layer 110. In use, the passageways 130 are positioned such that they mate or align with a standard 24, 96, 384, or 1536 pin replicator or multi-channel pipettor.

FIG. 2A also shows a confluent cellular layer of cells 220 grown onto the first membrane surface 104. By “confluent layer” it is meant that the cells are organized such that there exists a continuous layer of cells across the first membrane surface 104, where each cell abuts another cell, and there are no gaps or spaces between any two cells. This is otherwise known as “lawn growth”. Therefore, the confluent cellular layer of cells 220 forms a substantially planar cellular layer on the first membrane surface 104. Thus, for anything to permeate or diffuse through the transcellular testing device 200A, it must transport or diffuse through the confluent cellular layer of cells 220.

Because different skin locations provide different levels of permeability to foreign substances, such as ocular tissue compared to the epidermis on one's arm, the rate of transcellular diffusion varies accordingly. Therefore, transcellular testing of a drug compound must simulate the location in which the drug will be delivered. To do this, the thickness of the confluent cellular layer of cells 220 and the type of cells used to generate the confluent cellular layer of cells 220 are varied with respect to the test being performed. A suitable example of the cell type used in the confluent cellular layer of cells 220 is Caco 2 cells from the epithelial cell line. Other examples of cell types used in the confluent cellular layer of cells 220 include epithelial Madin-Darby canine kidney (MDCK) cells and human epidermal keratinocytes (HEK).

The confluent cellular layer of cells 220 is grown directly onto the first membrane surface 104 (as described in detail with respect to FIG. 3) and the cells of the confluent cellular layer of cells 220 attach to the pores of the membrane 102, as described above, thereby anchoring the confluent cellular layer of cells 220 to the membrane 102. It is preferable that the confluent cellular layer of cells 220 is a monolayer of cells.

The transcellular testing device 200A also preferably includes at least one donor substance 240 positioned directly onto the confluent cellular layer of cells 220. The donor substances 240 contain chemical substances such as drugs, inhibitors, activators, and excipients dissolved in a salt solution. The donor substance 240 is typically comprised of a cell compatible matrix, of the appropriate texture, and contains a water content equal to or greater than 70 percent. Suitable compounds include hydroxyethyl cellulose, hydroxypropyl cellulose, hydroxyethyl methacrylate, or the like. A suitable example of the composition of the donor substance 240 is a polymerized Mowiol® 28-99 made by Clariant International Ltd. of Switzerland, to which the test substances are added. It is also preferable that the salt concentration of the donor substance 240 is isotonic.

In a preferred embodiment of the invention there are multiple, or an array of donor substances 240. Each donor substance 240 preferably substantially aligns with a passageway 130 on the second membrane surface 106. In this configuration, each donor substance 240 has roughly the same diameter as the first opening 135, the second opening 136, and the passageway 130.

FIG. 2A also shows the receivers 250. In a preferred embodiment the receivers 250 are fluid droplets containing a balanced salt solution. In use, the receivers 250 are deposited within the first opening 135 of each passageway 130. Each passageway 130 holds each receiver 250. In a preferred embodiment the salt solution of the receivers 250 is isotonic. Also, in a preferred embodiment there are 24, 96, 384, or 1536 receivers 250.

It is preferable that each combination of aligned donor substance 240 and passageway 130 forms a discrete independent testing unit 260 such that each discrete independent testing unit 260 can test a different drug, inhibitor, adhesive, and additive combination simultaneously. For example, in use, substances dissolved in the donor substances 240 diffuse out of the donor substances 240, transfer across the confluent cellular layer of cells 220 in the direction depicted by the arrow 280, permeate through the membrane 102, and into the receivers 250 located within each passageway 130. In this embodiment, each combination of donor substance 240, passageway 130, and receiver 250 forms a discrete independent testing unit 260.

FIG. 2B is another embodiment of a transcellular testing device 200B, according to another embodiment of the invention. In this embodiment the hydrophobic layer 110B passes through the second membrane surface 106B and is embedded into the membrane 102B. (Second hydrophobic layer surface 112B is embedded into membrane 102B.) The hydrophobic layer 110B penetrates between about 10 micrometers and about 1 mm, and more preferably about 0.1 mm. In use, this forms a pseudo well or channel that helps maintain each passageway 130 as a discrete independent testing unit 260. Because the hydrophobic layer 110B is embedded into the membrane 102B, the testing substances of one discrete independent testing unit 260 is less likely to mix with the testing substances of a neighboring testing unit.

FIG. 2C is yet another embodiment of a transcellular testing device 200C, according to yet another embodiment of the invention. In this embodiment the thickness of the hydrophobic layer 110C is increased as compared to the embodiment corresponding to FIG. 2A. It is preferable that this hydrophobic layer 110C is between about 5 mm and about 20 mm thick, and more preferably about 10 mm thick.

Due to the increased thickness the volume of the receiver 250C can be greater. In use, due to the larger volume of the receivers 250C, multiple samples can be withdrawn over time from each receiver 250C for analysis. In this embodiment, the transcellular testing device 200C is configured for transcellular testing over longer periods of time, such as days or weeks.

FIG. 2D is even another embodiment of a transcellular testing device 200D, according to even another embodiment of the invention. In this embodiment the hydrophobic layer 110D is relatively thick, as described with respect to FIG. 2C. It is preferable that this hydrophobic layer 110D is between about 5 mm and about 20 mm thick, and more preferably about 11 to about 13 mm thick. In use, the volume of the receivers 250D is increased due to the increased thickness of the hydrophobic layer 110D. Therefore, multiple samples can be withdrawn over time from the receivers 250D for analysis. In this embodiment, the transcellular testing device 200D is configured for transcellular testing over longer periods of time, such as days or weeks.

Also, in this embodiment, the hydrophobic layer 110D is embedded through the second membrane surface 106D and into the membrane 102D. (Second hydrophobic layer surface 112D is embedded into membrane 102D.) The hydrophobic layer 110D penetrates between about 0.01 mm and about 1 mm, and more preferably about 0.1 mm into the membrane 102D. In use, this forms a pseudo well or channel that helps maintain each passageway 130 as a discrete independent testing unit 260. In use, each donor substance 240 preferably contains a different testing substance that diffuses to its associated receiver 250D. Because the hydrophobic layer 110D is embedded into the membrane 102D, the substances being tested in one discrete independent testing unit 260 are less likely to mix with the testing substances of a neighboring testing unit.

FIG. 2E is a further embodiment of a transcellular testing device 200E, according to a further embodiment of the invention. In this embodiment, the hydrophobic layer 110E is coupled to the second membrane surface 106, and an additional hydrophobic layer 217E is coupled to the first membrane surface 104. The hydrophobic layer 110E defines an array of passageways 130 therethrough, as described above. Similarly, the additional hydrophobic layer 217E defines an array of additional passageways 131E, 132E, and 133E therethrough. The passageways 130 are substantially aligned with the additional passageways 131E, 132E, and 133E, such that a donor substance 240 flowing in the direction of the arrow 280 (parallel to the z-axis) passes through the additional passageway 131E, 132E, or 133E, then continues into the passageways 130. Thereby, a plurality of permeable channel or conduit like discrete independent testing units 260 is formed. The first and second membrane surfaces at the openings of each passageway 130 and each additional passageway 131E, 132E, and 133E remain untreated.

The confluent cellular layer of cells 221E, 222E, and 223E are grown on the first membrane surface 104 within each additional passageway 131E, 132E, and 133E. The confluent cellular layer of cells 221E, 222E, and 223E attaches to the membrane 102 at the opening in the additional hydrophobic layer 217E formed by the additional passageways 131E, 132E, and 133E. Therefore, in use, each of the additional passageways 131E, 132E and 133E, on the first membrane surface 104 has its own discrete confluent cellular layer of cells 221E, 222E, and 223E that fully covers each additional passageway 131E, 132E, and 133E.

It should be stressed that the second hydrophobic layer surface 112 remain free from any cell culture medium used to grow the confluent cellular layer of cells 220, as described with respect to FIG. 3. This is because cellular proteins will stick to the second hydrophobic layer surface 112 and alter the surface characteristics, making the layer semi hydrophilic, thereby, causing the samples in the receivers 250 to deviate from discrete fluid droplets within each passageway 130.

FIG. 2F is still another embodiment of a transcellular testing device 200F, according to still another embodiment of the invention. This embodiment is similar to that of FIG. 2E except the hydrophobic layer 110F and the additional hydrophobic layer 217F are both embedded into the membrane 102F, as described with respect to FIG. 2B. In this configuration there is a channel formed by the hydrophobic layer 110F and the additional hydrophobic layer 217F, such that substances are less likely to migrate from one discrete independent testing unit 260 to a neighboring discrete independent testing unit. Furthermore, in this embodiment, the hydrophobic layer 110F is between about 5 mm and about 20 mm thick, and more preferably about 11 to about 13 mm thick. In this configuration, the receivers 250 preferably contain a balanced salt solution. Also, in this configuration, the receivers 250 contain sufficient solution to sustain prolonged testing and the extraction of multiple samples for analysis as described with respect to FIG. 2C.

FIG. 3 is a flow chart of the method 300 for making and using a transcellular testing device of the present invention. Once a membrane has been provided, as described above, a hydrophobic layer 110 (FIG. 2A-2F) is coupled to the second membrane surface 106 (FIG. 2A-2F), at step 310. The hydrophobic layer 110 defines passageways 130 (FIG. 2A-2F) therethrough, such that as the hydrophobic layer 110 (FIG. 2A-2F) is coupled to the second membrane surface 106 (FIG. 2A-2F), areas of the membrane 102 (FIG. 2A-2F) do not couple with the hydrophobic layer 110 (FIG. 2A-2F).

In an alternative embodiment, separate hydrophobic layers are coupled to opposing surfaces of the membrane 102. In this configuration, each hydrophobic layer defines passageways therethrough. Once the hydrophobic layers are coupled to their respective surface of the membrane, the passageways on one surface of the membrane 102 are in substantial alignment with the passageways on the opposing surface of the membrane.

In yet another embodiment, also at step 310, coupling of the hydrophobic layer includes embedding the hydrophobic layer into the membrane. In this configuration, the hydrophobic layer 110 penetrates at least partially through the membrane surface and is embedded into the membrane. The hydrophobic layer 110 penetrates between about 0.01 and about 1 mm, and more preferably about 0.1 mm. In use, this forms a pseudo well or channel that helps maintain each passageway 130 in a discrete independent testing unit 260 (FIG. 2A-2F).

In another embodiment, also at step 310, the separate hydrophobic layers coupled to opposing surfaces of the membrane both penetrate through the membrane surfaces, respectively, and embed into the membrane. For example, the hydrophobic layer 110 (FIG. 2F) is embedded through the second membrane surface 106 and embedded into the membrane 102. The additional hydrophobic layer 217 (FIG. 2F) penetrates the first membrane surface 104 and is embedded into the membrane 102. Both hydrophobic layers are preferably embedded between about 0.01 mm and about 1 mm, and more preferably about 0.1 mm into the membrane 102.

At step 320, a confluent cellular layer of cells 220 (FIG. 2A-2F) is grown on at least one surface of a membrane 102. The confluent cellular layer of cells is a continuous layer covering substantially the entire membrane surface. The cells of the confluent cellular layer of cells abut one another such that there are no gaps between the cells. Therefore, for donor substances to pass through the confluent cellular layer of cells, the donor substance must pass through at least one cell.

A suitable example of growing a confluent cellular layer of cells 220 on a surface of the membrane 102 is described in relation to steps 322, 324, 326, and 328. At step 322, the membrane 102 (FIG. 2A-2F) is placed second membrane surface 106 down on a bottom plate (not shown) and clamped around the perimeter with an open top frame (not shown). Because the open top frame (not shown) is simply a perimeter frame, the membrane clamped between the open top frame (not shown) and the bottom plate (not shown) is accessible. Therefore, the first membrane surface 104 is left exposed. The exterior junction between the open top frame and the bottom plate is then sealed, such that no substances deposited within the open top frame (not shown) can leak out between the open top frame and the bottom plate (not shown). For example, suitable sealing of the juncture between the open top frame and the bottom plate is by parafilm with or without a tape overlay.

Next, a cell suspension in standard growth medium containing serum is seeded onto the exposed first membrane surface 104 (FIG. 2A-2F), at step 324. In a preferred embodiment, the top frame (not shown) acts as a reservoir for the cell suspension and growth medium. The combination top frame, bottom plate, clamped membrane 102, and cell suspension is then placed within a sterile container and incubated, at step 326. In a preferred embodiment the cells of the cell suspension are incubated at about 37 degrees Celsius in approximately a 5 to 10 percent carbon dioxide atmosphere. The length of time for incubating varies depending on the time for attachment of the specific cell type to the membrane.

Following incubation, at step 328, the membrane 102 is removed from between the top frame (not shown) and the bottom plate (not shown) and floated, cell-side down (hydrophobic layer 110 side up) in a dish containing complete cellular growth medium. The cell cultures are maintained in this configuration with periodic replenishment of cell growth medium until the cellular layer is post-confluent, i.e., a confluent cellular layer of cells is grown, at step 328. The rate of cell growth varies considerably with cell type and can take, for example, from 1 day to 30 days to obtain a post-confluent cellular layer.

It is stressed that during steps 324, 326, and 328 the second hydrophobic layer surface 112 (FIG. 2A-2F) remains free from any cell culture medium used to grow the confluent cellular layer of cells 220. This is because cell culture medium contains serum and other proteins that adhere to the second hydrophobic layer surface 112 (FIG. 2A-2F) and alter its characteristics, rendering the hydrophobic nature of the material hydrophilic and therefore, unsuitable to maintain discrete receivers 250 (FIG. 2A-2F), as described above.

The membrane 102 (FIG. 2A-2F), with the attached hydrophobic layer 110 (FIG. 2A-2F) and confluent cellular layer of cells 220 (FIG. 2A-2F) is then removed from the cell growth medium and positioned to receive at least one donor substance 240 (FIG. 2A-2F), at step 330. Drug compounds to be tested or combinations of drugs, adhesives, enhancers, inhibitors, or the like are selected and incorporated into the donor substances 240 (FIG. 2A-2F).

Donor substances 240 (FIG. 2A-2F) are then deposited onto the confluent cellular layer of cells 220 in substantial alignment with each passageway 130. Receivers 250 (FIG. 2A-2F), as described above, are then positioned within the passageways 130 (FIG. 2A-2F), at step 340. Transcellular testing is then conducted with the transcellular testing device 200A-200F, at step 350.

At step 352, the substances dissolved in the donor substances diffuse out of the donor substances 240 (FIG. 2A-2F), through the confluent cellular layer of cells 220 (FIG. 2A-2F), the membrane 102 (FIG. 2A-2F), and into the receivers 250 (FIG. 2A-2F). Samples are then retrieved from the receivers 250 (FIG. 2A-2F), at step 354. In a preferred embodiment, at step 354, aliquots from the receivers 250 (FIG. 2A-2F) are retrieved for analysis. In an alternative embodiment, at step 354, the entire quantity of each of the receivers 250 (FIG. 2A-2F) is retrieved for analysis.

Next, the samples from the receivers 250 (FIG. 2A-2F) are analyzed, at step 356, to determine the substance concentration in the receivers 250 (FIG. 2A-2F) at definite time intervals to determine the concentration of donor substances retrieved from the receivers 250 and the flux rate for the particular substance tested through the particular confluent cellular layer of cells 220 (FIG. 2A-2F) used in the particular experiment protocol. The concentration can be determined using one or more of many techniques known to those skilled in the art including, for example, UV spectroscopy and HPLC.

Although the present invention has been described in considerable detail with reference to certain preferred embodiments, other embodiments are possible. Therefore, the spirit and scope of the appended claims should not be limited to the description of the preferred embodiments contained herein. Modifications and variations of the invention described herein will be obvious to those skilled in the art from the foregoing detailed description and such modifications and variations are intended to come within the scope of the appended claims.

1-28. (canceled)

29. A transcellular testing device, comprising:
(a) a membrane having a first membrane surface opposing a second membrane surface, wherein said first membrane surface is configured for cellular adhesion; and
(b) a hydrophobic layer having a first hydrophobic layer surface opposing a second hydrophobic layer surface wherein said second hydrophobic layer surface is coupled to said second membrane surface, and wherein said hydrophobic layer comprises at least one discrete independent testing unit having;
(i) a first opening in said first hydrophobic layer surface;
(ii) a second opening in said second hydrophobic layer surface; and
(iii) a passageway defined by said hydrophobic layer between said first opening and said second opening.

30. The transcellular testing device of claim 29, wherein said second hydrophobic layer surface is adhered to said second membrane surface.

31. The transcellular testing device of claim 29, wherein said second hydrophobic layer surface is heat sealed to said second membrane surface.

32. The transcellular testing device of claim 29, further comprising an array of discrete independent testing units.

33. The transcellular testing device of claim 29, wherein said membrane is hydrophilic.

34. The transcellular testing device of claim 29, wherein said passageway exposes an area of said second membrane surface which is hydrophilic.

35. The transcellular testing device of claim 29, wherein a confluent cellular layer of cells is coupled to said first membrane surface.

36. The transcellular testing device of claim 35, further comprising a donor substance disposed on said confluent cellular layer of cells in substantial alignment with said passageway.

37. The transcellular testing device of claim 35, wherein said confluent cellular layer of cells is a monolayer of cells.

38. The transcellular testing device of claim 35, wherein said confluent cellular layer of cells is selected from the group consisting of: Caco 2, MDCK, and HEK cells.

39. The transcellular testing device of claim 36, wherein said donor substance contains an additive selected from the group consisting of: drug, inhibitor, adhesive, solvent, carrier, and transport enhancer.

40. The transcellular testing device of claim 29, wherein said membrane has a length of about 85.5 mm, and a width of about 127.8 mm, such that said membrane is configured to cooperate with a standard pin replicator or a multi-channel pipettor of a high density array microtiter plate.

41. The transcellular testing device of claim 29, further comprising a receiver within said passageway.

42. The transcellular testing device of claim 29, further comprising an additional hydrophobic layer coupled to said first membrane surface, wherein said additional hydrophobic layer defines at least one additional passageway therethrough in substantial alignment with said passageway.

43. The transcellular testing device of claim 42, wherein said additional passageway exposes an area of said first membrane surface which is hydrophilic.

44. The transcellular testing device of claim 42, wherein a confluent cellular layer of cells is bound to said first membrane surface.

45. A method of transcellular testing, comprising:
(a) providing a membrane having a first membrane surface opposing a second membrane surface, wherein said first membrane surface is configured for cellular adhesion; and
(b) coupling a hydrophobic layer to said second membrane surface wherein said hydrophobic layer defines at least one passageway therethrough.

46. The method of transcellular testing of claim 45, further comprising growing a confluent cellular layer of cells on said first membrane surface of said membrane.

47. The method of transcellular testing of claim 45, further comprising:
(a) seeding cells onto said first membrane surface;
(b) incubating said cells at about 37 degrees Celsius in about a 5 to 10 percent carbon dioxide atmosphere; and
(c) maintaining the membrane in a container comprising complete cellular growth medium until confluent cellular layer of cells is grown on said first membrane surface.

48. The method of transcellular testing of claim 45, further comprising depositing at least one donor substance onto said confluent cellular layer of cells in substantial alignment with said passageway.

49. The method of transcellular testing of claim 45, further comprising placing a receiver within said passageway defined by said hydrophobic layer.

50. The method of transcellular testing of claim 49, further comprising retrieving samples from said receiver at predetermined time intervals.

51. The method of transcellular testing of claim 50, further comprising analyzing said retrieved samples from said receiver at predetermined time intervals for determination of said donor substances.

52. The method of transcellular testing of claim 51, further comprising calculating a flux rate for said retrieved samples from said receiver.

53. The method of transcellular testing of claim 46, further comprising:
(a) selecting substances to test for diffusion through a cellular layer;
(b) combining test substances in a donor substance;
(c) positioning said donor substance on said confluent cellular layer of cells wherein said donor substance is positioned in substantial alignment with said passageway;
(d) receiving samples from receivers positioned within said passageway; and
(e) analyzing said samples to determine a diffusion rate across said confluent cellular layer of cells.
